(19) United States
(12) Patent Application Publication
(10) Pub. No.: US 2009/0232745 A1
(43) Pub. Date: Sep. 17, 2009

(54) LICORICE LOLLIPOP THAT INHIBITS DENTAL CARIES FORMATION

(75) Inventor: MAXWELL H. ANDERSON, Sequim, WA (US)

Correspondence Address: Weaver Austin Villeneuve & Sampson LLP, P.O. BOX 70250, OAKLAND, CA, 94612-0250, US

(73) Assignee: INTELLIHERB, INC., Inglewood, CA (US)

(21) Appl. No.: 12/404,119

(22) Filed: Mar. 13, 2009

Related U.S. Application Data
(60) Provisional application No. 61/036,792, filed on Mar. 14, 2008.

(51) Int. Cl. A61K 9/12; A61K 8/97; A61Q 11/00

(52) U.S. Cl. 424/45; 424/58

(57) ABSTRACT

This invention pertains to the discovery that certain extracts of licorice, particularly when formulated as a candy or lollipop or other means that provides an extended release (e.g., greater than about 4 minutes) to the oral cavity significantly inhibits the formation of dental caries in a human subject.

### CROSS-REFERENCE TO RELATED APPLICATIONS

This application claims benefit of and priority to U.S. Ser. No. 61/036,792, filed on Mar. 14, 2008, which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention pertains to the fields of dentistry and preventative medicine. In particular, in certain embodiments, this invention provides a lollipop that inhibits the formation of dental caries.

### BACKGROUND OF THE INVENTION

The genus Glycyrrhiza consists of about 30 species (Nomura et al. (2002) Pure Appl. Chem. 74: 1199-1206) of which some have been used by humans for over 4000 years (Wang and Nixon (2001) Nutr. Cancer, 39: 1-11). Licorice is the name typically applied to the roots and stolons of Glycyrrhiza species (fabaceae). Glycyrrhiza uralensis Fisch. ex DC. (Chinese name “Gancao”, or Chinese licorice) is one of the most frequently used traditional medicines in mainland China and in some other countries as well. Over the past decades, many research groups have investigated its chemical constituents (see, e.g., Shibano et al. (1997) Heterocycles, 45: 2053-2060; Hatano et al. (1998) Phytochemistiy 47: 287-293; Hatano et al. (2000) Phytochemistiy 55: 959-963; Fukai et al. (1989) Heterocycles, 29, 1369-1378; Fukai et al. (1993) Heterocycles, 36: 2565-2576; Kiuchi et al. (1990) Heterocycles 31: 629-636) and biological activities (Nomura et al. (2002) Pure Appl. Chem. 74: 1199-1206; Fukai et al. (2002) Fitoterapia, 73: 536-539; Fukai et al. (2004) Planta Med. 70: 685-687; Tsukiyama et al. (2002) Antimicrob. Agents Chemother. 46: 1226-1230; Hatano et al. (2000) Chem. Pharm. Bull. 48: 1286-1292; Hsieh and Wu (2002) Int. J. Oncol., 20: 583-588; He et al. (2006) J. Nat. Prod., 69: 121-124).

Various chemical studies have led to the identification of about 100 phenolic compounds, many of which are isoprenoid-substituted phenols (Hatano et al. (1998) Phytochemistry 47: 287-293; Hatano et al. (2000) Phytochemistry, 55: 959-963). Most of these isoprenoid-substituted flavonoids are isoflavans with oxygen substituted at the C-5 position.

Certain licorice extracts have been used to treat various diseases in respiratory system, gastrointestinal system, cardiovascular system, urogenital system, skin, eye etc (see, e.g., Fiore et al. (2005) J Ethnopharmacol. 99(3): 317-324; Shibata (2000) Yakugaku Zasshi. 120(10): 849-862). Many interesting chemical molecules have been identified from the herb that are associated with various therapeutic effects.

### SUMMARY OF THE INVENTION

This invention pertains to the discovery that certain extracts of licorice, particularly when formulated as a candy or lollipop or other means that provides an extended release (e.g., greater than about 4 minutes) to the oral cavity significantly inhibits the formation of dental caries in a human subject.

This invention pertains to the discovery that certain extracts of licorice, particularly when formulated as a candy or lollipop or other means that provides an extended release (e.g., greater than about 4 minutes) to the oral cavity significantly inhibits the formation of dental caries in a human subject.

Accordingly, in certain embodiments, compositions are provided for reducing the incidence and/or severity of dental caries in a subject (e.g., a human). In various embodiments the composition comprises a lollipop or hard candy, where the lollipop or hard candy is substantially sugar free (or substantially free of carogenic sweeteners), and the lollipop or hard candy comprises an extract from the root of Glycyrrhiza spp., where the extract comprises a glycyrrhizol and/or a glycyrrhizin. In certain embodiments the lollipop or hard candy is a lollipop. In certain embodiments the lollipop or hard candy comprises a starch, and/or dextrin, and/or erythritol, and/or meso-erythritol, and/or malt, a flavoring, a sweetener, and the extract. In certain embodiments the sweetener is noncariogenic. In certain embodiments the sweetener is a sugar alcohol. In certain embodiments the sweetener is an artificial sweetener. In certain embodiments the sweetener is selected from the group consisting of stevia, aspartame, saccharin, acesulfame k, sucralose, and neotame. In certain embodiments the lollipop or hard candy comprises: hydrogenated starch hydrolysate; citric acid; flavoring; and acesulfame potassium. In certain embodiments the lollipop or hard candy further comprises a coloring agent and/or a preservative. In certain embodiments the extract comprises substantially purified glycyrrhizol A, and/or substantially purified glycyrrhizol B, and/or substantially purified 6,8-diisoprenyl-5,7,4′-trihydroxyisoflavone. In certain embodiments the extract consists essentially of substantially purified glycyrrhizol A and/or substantially purified glycyrrhizol B, and/or substantially purified 6,8-diisoprenyl-5,7,4′-trihydroxyisoflavone (e.g., it has the activity of substantially purified glycyrrhizol A and/or substantially purified glycyrrhizol B, and/or substantially purified 6,8-diisoprenyl-5,7,4′-trihydroxyisoflavone). In certain embodiments each lollipop or hard candy contains from about 2 mg to about 200 mg of the licorice extract. In certain embodiments the lollipop or hard candy contains from about 2 mg, 5 mg, or 10 mg to about 100, 50, 25, 20, or 15 mg of the extract. In certain embodiments each lollipop or hard candy contains from about 2 mg to about 50 mg, 30 mg, 25 mg, 20 mg, or 15 mg of the extract. In certain embodiments the hard candy or lollipop is formulated to dissolve in about 1 or 2 minutes to about 15 minutes in a human mouth. In certain embodiments is formulated to take at least about 2 minutes, 3 minutes, 4 minutes, or 5 minutes to dissolve in a human mouth. In certain embodiments the licorice extract is an extract produced as described herein and in the claims.

Also provided are methods of reducing the incidence or severity of dental caries in a human. The methods typically involve administering to the human a composition that delivers to the oral cavity of the human a licorice extract that inhibits dental carie formation or severity, where the delivery is for a period of at least about 4 minutes. In certain embodiments the licorice extract comprises a glycyrrhizol and/or a glylcyrrhizin and/or 6,8-diisoprenyl-5,7,4′-trihydroxyisoflavone. In certain embodiments the period is at least 1 minute. In certain embodiments the period is at least 5 minutes. In certain embodiments the period ranges from about 5 minutes to about 15 minutes. In certain embodiments the extract comprises a glycyrrhizol extracted from the root of Glycyrrhiza spp. In certain embodiments the extract comprises or consists essentially of a substantially purified glycyrrhizol A and/or substantially purified glycyrrhizol B, and/or or substantially purified 6,8-diisoprenyl-5,7,4′-trihydroxyisoflavone. In certain embodiments about 2 mg, 5 mg, or 10 mg, 15 mg, or 20 mg to about 100, 50, 25, 20, or 15 mg of the extract is delivered to the oral cavity in a single administration (e.g., consumption of a single lollipop or hard candy). In certain embodiments the composition comprises a lollipop containing a glycyrrhizol. In certain embodiments the composition comprises a lollipop or hard candy as described above or below herein.

In certain embodiments methods of producing an extract of licorice that inhibits the formation of dental caries by Streptococcus mutans are provided. In certain embodiments the methods typically involve providing root of Glycyrrhiza spp.; performing an alcohol extraction of the root; and drying the extract to produce an extract that has activity against S. mutans. In certain embodiments the activity is comparable to or equivalent to the activity of substantially purified glycyrrhizol A. In certain embodiments the root is cut to pieces having an average thickness of about 2 mm or less, or about 1 mm or less. In certain embodiments the root is grown and harvested in inner Mongolia. In certain embodiments the alcohol extraction comprises a first extraction combining the root with 95% food grade alcohol (e.g., ethanol) in about a 1:8 ratio. In certain embodiments the root/ethanol mixture is incubated for about 4 hours, about 8 hours, about 12 hours, about 24 hours, about 48 hours, or about 72 hours or longer. In certain embodiments the method further comprises the ethanol, and performing a second extraction by adding fresh 95% food grade alcohol (e.g., ethanol) in about a 1:8 ratio and incubating again. In certain embodiments the alcohol is dried to produce a substantially dry powder extract. In certain embodiments the method does not comprise solvent/solvent partitioning using an organic solvent or a solvent/solvent partitioning using an inoorganic solvent.

In certain embodiments compositions for reducing dental caries in a human subject are provided. The compositions typically comprise a licorice extract that inhibits dental caries formation. In certain embodiments the extract is an extract produced as described herein. In certain embodiments the licorice extract is contained in a product selected from the group consisting of toothpaste, mouthwash, a tooth whitening strip or solution, dental floss, a toothpick, a toothbrush bristle, an oral spray, an oral lozenge, and an aerosolizer for oral application.

### DEFINITIONS

The term “substantially purified” when used with respect to Glycyrrhiozl A indicates that the compound/extract usually has anti-S. mutans activity at 10-30 microgram per ml as compared to pure Glycyrrhizol A which has anti-S. mutans activity at 1 microgram per ml.

A “licorice extract” refers to a composition comprising one or more chemical compounds extracted from the licorice plant Glycyrrhiza spp. In certain embodiments the licorice extract is an extract produced as described herein. In certain embodiments the extract comprises a glycyrrhizol (e.g., glycyrrhizol A and/or glycyrrhizol B) and/or glycyrrhizin. In certain embodiments the glycyrrhizol A and/or glycyrrhizol B are substantially purified.

The term “lollipop” refers to a piece of hard candy, often approximately spherical, ovoid, or disk-shaped, attached to a stick or “handle”.

The term “reducing the incidence of dental caries” refers to inhibiting the formation of and/or reducing the severity of, or magnitude of, dental caries.

The term “substantially sugar-free” indicates that the composition is substantially free and preferably wholly free of caries-forming sugars. In various embodiments sugar alcohols can and other similar agents can be used to form the base and sweetener.

The term “noncariogenic” with reference to a sweetener indicates that the sweetener has a reduced ability or cannot be fermented by cariogenic microorganisms that are present in the oral flora. In various embodiments the sweetener cannot be fermented to harmful acids. Illustrative noncariogenic sweeteners include but are not limited to, acesulfame k; alitame; aspartame; cyclamate; erythritol; meso-erythritol, neotame; saccharine; stevioside; sucralose, isomaltose, and tagatose. It is noted that high intensity sweeteners such as aspartame, sucralose, and neotame all have a more enduring sweetness than sucrose. Accordingly, in certain embodiments, where the sweeteners are used in conjunction with an acidulant, they can, optionally, be used with malic rather than citric acid. Malic Acid has a more persistent sourness than citric acid and therefore complements these sweeteners, even when used as a secondary acidulant

### BRIEF DESCRIPTION OF THE DRAWINGS

FIG. 1 shows the formula of Glycyrrhizol A.

FIG. 2 shows the formula of Glycyrrhizol B.

FIG. 3 illustrates an HPLC for of the licorice extract.

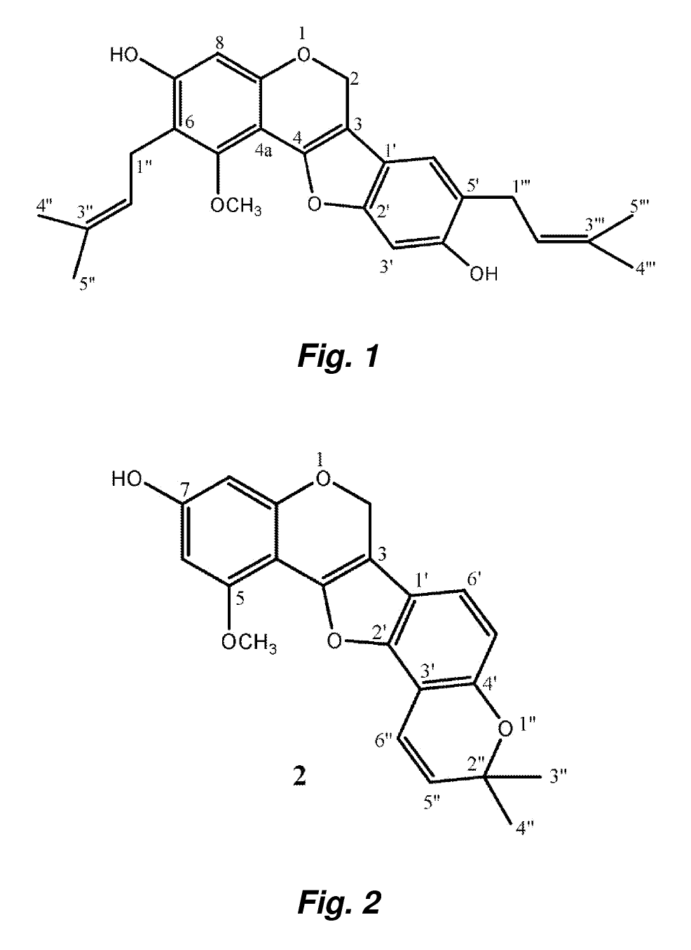

### DETAILED DESCRIPTION

This invention pertains to the identification of novel licorice extracts that show good efficacy against Streptococcus mutans (S. mutans) and that are effective to inhibit dental caries formation in human subjects, particularly when administered as a lollipop and/or hard candy (e.g., a lozenge).

It was a particularly surprising discovery that when administered in a lozenge form, the licorice extract(s) are effective to inhibit the adherence and/or growth and/or proliferation of S. mutans in the oral cavity of the subject consuming the lozenge. Moreover, it is believed that a relatively short period of administration (e.g., over a period of hours or days, preferentially up to about 1 or 2 weeks) results in a prolonged diminution of the S. mutans population in the oral cavity for a period of up to 3 months, preferably 6 months, more preferably up to 9 months, and most preferably up to one year. Since the extract needs to be administered only intermittently, it is believed that bacterial resistance is unlikely to develop.

Without being bound to a particular theory, it is believed that selective/preferential reduction of S. mutans in the oral cavity permits other (non-caries forming) bacterial species to proliferate to a level that slows the re-establishment of the S. mutans population.

In addition to the lozenge formulation(s), effective methods are provided for obtaining high activity licorice extracts with good consistency. The methods are well suited to the commercial manufacture of the licorice extract described herein.

### Licorice Extract Production.

It was a surprising discovery that different licorice extracts can have significantly different activities depending on the extraction method and/or source of licorice. In addition it was a surprising discovery that glycyrrhizin which is believed to have little antimicrobial effect can inhibit formation of dental caries by inhibiting bacterial adherence.

Knowing that different licorice extracts have different bioactivities, we developed a novel extraction procedure that allows us to consistently obtain licorice extracts having high antimicrobial activity against Streptococcus mutans.

To achieve this effect, we have collected licorice roots from five different parts of China, Zhejiang, Jiangsu, Gansu, Xingjiang and Inner Mongolia provinces, and found that licorice roots from Inner Mongolia province have the best activity (the activity could vary up to 10 fold).

We cut licorice roots into different forms, used different extraction methods (such as water boiling and ethanol soaking) with different times and ratios, tried different filtration and drying methods. With different methods, the outcome of the active ingredients could vary by a factor of up to 5000 fold. Here is the method that consistently provides us the best results:

a) Licorice roots grown and harvested in Inner Mongolia province;

b) The licorice roots are cut into thin slices (about 1 mm);

c) 100 kilogram licorice root slices are mixed with 95% food grade ethanol in 1:8 ratio in an extraction container, and incubated at 37° C. for 72 h (these volumes can be scaled up proportionately);

d) After the first batch of ethanol is removed, more fresh 95% food grade ethanol is added to the extraction container in 1:8 ratio at 37° C. for 96 h;

The resulting ethanol solution (containing extracts) are filtered three times;

The extracts are then concentrated through ethanol evaporation

The concentrated extracts are then dried with a microwave “dry” distillation method, which is a combination of microwave heating and dry distillation, performed at atmospheric pressure. The final yield is about 4% of the raw weight of the licorice roots.

We also established an effective QA/QC system for every batch of licorice extract produced in large quantity.

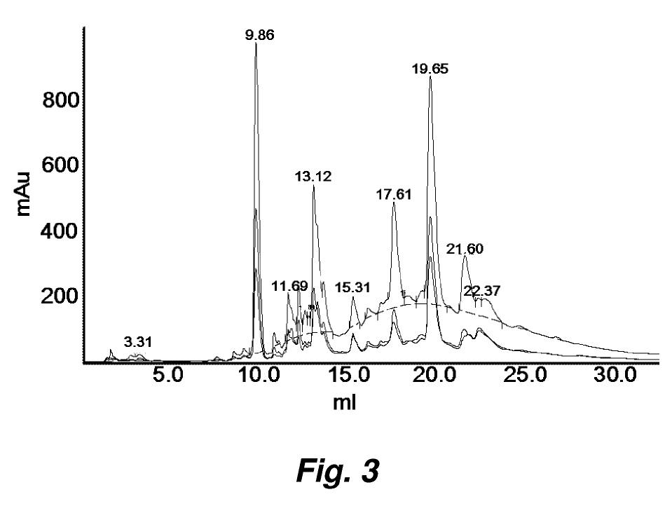

All extracts are assayed with HPLC (see, e.g., FIG. 3) for the amount of Glycyrrhizol A which proved to be a strong indicator of activity against dental caries. The HPLC conditions were:

Analysis was carried out with Amersham Biosciences HPLC Instrument. SOURCE™ 15RPC (4.6/100)

Flow rate: 0.5 mL/min

Elution system: H₂O (0.1% TFA, solvent A) and CH₃CN (0.1% TFA, solvent B)

Detector: UV 215, 260 and 280 nm

Eluting gradient: From 10% (B) to 100% (B) in 15 CV (column volume, ICV=1.662 mL).

Under these conditions, we can effectively detect glycyrrhizol amounts that are consistent with the biological efficacy studies.

In addition to chemical analysis, all batches, or statistically relevant samples of continuous production processes (e.g., 1/3600 candies; or 1/5000 candies; or 1/10,000 candies or the like) or statistically relevant samples of candy production batches (e.g., 1/10 batches; or 1/20 batches; or 1/50 batches), are examined for bioactivity. While the pure compound of glycyrrhizol A has anti-S. mutans activity at 1 microgram per ml, the mixed compound usually has anti-S. mutans activity at 10-30 microgram per ml.

### Lozenge Formulation.

We also developed a lozenge (e.g., lollipop) as a unique delivery device to deliver antimicrobial compounds of licorice extracts to the oral cavity. We tested many delivery mechanisms such as water solution, gel, toothpaste, etc. Finally we decided to use sugarless lollipop since we can deliver the active ingredient through a mechanism that causes the problem. In addition, this facilitates deliver to the target population without demanding behavioral changes.

The lollipop is an acceptable, safe delivery mechanisms that is welcomed by children, the elderly population, and other needy populations.

It was also a surprising discovery that lollipops provide a particularly effective delivery profile. People usually will chew hard or soft candies and finish them typically in less than 3 minutes. Chewing gum will release all major contents in the first 30 seconds. In contrast, it typically takes 5-10 minutes to dissolve a lollipop, which provides optimal killing kinetics, as described below:

a. In vitro time-killing data against S. mutans

| | |
|---|---|
| a. 30 sec | 31% killing |
| b. 60 sec | 41% killing |
| c. 5 min | 67% killing |
| d. 10 min | 85% killing |

We formulated a licorice-extract-containing lollipop that shows good efficacy, stability and safety. Contracted through a candy making company, we made different candies with various concentrations of licorice extracts in various forms that are mixed with various flavors. For example, we tried 5, 15, 25 mg herbal extracts (per lollipop). We also tried dry powder, or extracts dissolved in water or ethanol. We tried to add herbal powders at different stages of candy making process and we tried many different flavors such as mint, orange etc.

In various embodiments the licorice extract is simply combined with a sugar-free candy base to form a lollipop or other hard candy, that preferably delivers a dose profile of the licorice extract as described above.

Sugar-free candy bases are well known to those of skill in the art. For example, Japanese patent publication JP-B-56-18180 discloses a sugar-free candy base comprising erythritol. U.S. Pat. No. 4,883,685, which is incorporated herein by reference for the compounds and formulations disclosed therein, discloses a sugar-free candy base comprising meso-erythrito. A basic formula for a lactitol-based hard candy comprises lactitol, maltitol syrup, acid, sweetener, color, and flavor. The lactitol can be dissolved into the syrup and no additional water is required.

Other sugar-free candy bases are described for example in WO/1997/003569 (PCT/US1996/011620) which is incorporated herein by reference for the compositions and methods described therein. These bases comprise mainly a substantially non-hygroscopic sugar alcohol candying solution. This typically comprises a commercially available, economically satisfactory, sugar alcohol that, when cooked, forms a substantially non-hygroscopic hard, cooked, product. Illustrative sugar alcohols include, but are not limited to, isomalt (for instance the commercial product Isomalt M supplied by Palatint Sussungsmitel GmbH of Mannhein Germany), sorbitol, xylitol, lactitol, mannitol and polydextrose. Other hydrogenated monosaccharides and disaccharides can be used and mixtures of hydrogenated saccharides and polysaccharides, such as hydrogenated glucose syrup, can also be used. Mixtures are also suitable. In certain embodiments the composition contains at least 50% by weight (dry matter after cooking) of the named sugar alcohols and usually at least 80% and frequently at least 90% by weight of the named sugar alcohols. Usually the only components of the composition that, optionally, are not cooked along with sugar alcohols (and trace water remaining from the casing composition) are generally colorings, acidifying agents and flavorings and/or the licorice extracts described herein. In certain embodiments the total amount of such materials is usually below 10% by weight, and generally below 5% by weight of the cooked composition. In certain embodiments the formulation provides approximately 10 mg licorice extract per approximately 9 grams of candy.

The preferably amount of licorice extract ingredient in certain embodiments is described below. The amount of preservative and/or colorants, and/or flavorings can be conventional for hard candies. The relevant additives such as flavors, colorants or other active ingredients can be present in conventional amounts.

In various embodiments the candy can be made by appropriate combination of conventional candy-making process steps. As is known, a hard candy material can be made by cooking an aqueous solution of the sugar alcohol to provide a syrup, generally referred to as a candying solution, having an adequately low water content. Generally the water content of the candying solution must be below 3%, preferably below 2%. The candying solution is then allowed to cool, with consequential crystallization and the formation of the hard candy component.

The non aqueous components of the aqueous solution which is subjected to cooking are provided by the sugar alcohol or alcohols and the desired amount, usually not more than 5%, flavoring, colorants, acidifiers. Alternatively some or all of the additives may be added to the candying solution after cooking and before cooling and crystallization.

When the cooking is conducted in bulk, it is conducted to a temperature selected according to the materials being used and the process conditions. Often the cooking is being conducted on a microfilm cooker, for instance at temperatures of 130° C. to 170° C.

In one illustrative embodiment, a sugar alcohol solution composition is formed from 67% w/w isomalt, and 33% w/w water and is cooked, e.g., in a microfilm cooker to a temperature of 160° C. at which time the resultant candying solution has a water content of about 2% w/w. To this casing candying solution can be added added: flavoring (e.g., ˜0.3% w/w to ˜2% w/w), optionally citric acid (e.g., ˜1% w/w), optionally colorant, and the licorice extract.

One preferred formulation that yielded good efficacy was 15 mg licorice extract powder added to orange flavored sugar-free candy syrup during the cooling process.

In this embodiment, a typical lollipop contained: Hydrogenated Starch Hydrolysate (HSH), Citric Acid, Natural Orange Flavor, Colors FD&C Yellow #6, artificial sweetener (Acesulfame Potassium), and about 15 mg extract of Glycyrrhiza uralensis.

In certain embodiments, there were 50 lollipops per pound and 15 mg in each lollipop so, with 453.6 grams/pound that provided 15 mg in 9.07 grams of candy per lollipop (˜0.16% w/w). It is recognized, however, that depending on the formulation of the candy base, size of the lozenge, and the like the amount of licorice extract may be varied to optimize activity. Accordingly, in certain embodiments, the extract ranges from about 0.01% w/w to about 1% w/w, preferably about 0.05% w/w to about 0.8% w/w, more preferably from about 0.1% to about 0.4% w/w, 0.3% w/w or 0.2% w/w and, in certain embodiments, from about 0.1% to about 0.3% w/w.

In another embodiment, a typical lozenge contained: Isomalt, Citric Acid, Natural Orange Flavor, Colors FD&C Yellow #6, artificial sweetener (Acesulfame Potassium), and about 15 mg extract of Glycyrrhiza uralensis.

In certain embodiments, there were 90 lozenges per pound and 8.3 mg Glycyrrhiza uralensis extract on each lozenge so, with 453.6 grams/pound that provided 8.3 mg in 5 grams of candy per lozenge.

Advantages of lollipop/hard candy formulation included:

1) A 15 mg lollipop has good stability as shown by the HPLC profile of the active component which is unchanged after 6 month of storage at 25° C.;

2) The killing ability remains after candy making profile (a 10 min period of high temperature);

3) The herbal lollipop retains >95% killing activity after 6 months of storage;

4) The killing activity of the extracts, assayed by in vitro time-killing assays, is unchanged after 6 month at 25° C.;

5) The lollipop has no safety issues;

6) Genome toxicity with Ames test showed that no toxicity was detected;

7) Cellular toxicity with epithelial cells, immune cells, liver and kidney cells, no toxicity is detected;

8) Animal toxicity with oral and IV injection of 200 mg/ml herbal extract per mouse showed no detectable toxicity.

In addition to the in vitro efficacy studies above, we have conducted pilot human studies. Based on three small pilot studies (a total of 24 subjects), the subjects with high S. mutans (over 500,000 S. mutans per ml saliva) exhibited a dramatic reduction of S. mutans from 5 fold to 500 fold. No human subjects using lollipops reported any adverse effects of using this lollipop.

In view of the foregoing, in certain embodiments, this candy lozenge (e.g., lollipop) is provided that contain an extract from the root of Glycyrrhiza spp., wherein said extract comprises a glycyrrhizol and, on typical consumption deliver this extract in an amount sufficient to inhibit adhesion and/or growth and/or proliferation of S. mutans in an oral cavity.

### Other Formulations.

As a dry powder with consistent antimicrobial activity with good QA/QC procedures, the licorice extracts described herein can be used for many other applications. These include, but are not limited to expanded dental applications, e.g., tea, toothpick coatings, dental floss coatings, toothpaste, gel, mouthwash, varnish, even professional dental products.

Also, the licorice extract can kill other pathogenic bacteria. For example, the extract has activity against the bacteria involved in both periodontitis and halitosis, and is useful in treating/mitigating these conditions. In addition, it can also can kill bacteria involved in lung infections and ear infections that may originate from oral cavity. Thus the use of the active extract(s) in ear washes, aerosol inhalers, mouth washes, and the like is also contemplated.

In certain embodiments, methods of treating or reducing the incidence, duration, or severity of periodontal disease are provided. The methods can include applying to the gingival crevice or periodontal pocket a composition comprising a licorice extract (e.g., glycyrrhizol composition) as described herein with a carrier/stabilizing agent. In the composition applied, the carrier/stabilizing agent can provide retention, tissue penetration, deposition and sustained release of the a licorice extract for reducing the population of specific bacterial species within a periodontal biofilm and associated tissues. In certain embodiments, the carrier agent provides penetration and retention into the gingival crevice or periodontal pocket and associated tissues with sustained release of the licorice extract to enhance the reduction in population of select bacteria within the gingival tissue and dentinal tubule tissue.

In various embodiments, carrier agents can include, but are not limited to polylactide, polyglycolide, polylactide-co-glycolide, polycaprolactone, cellulosic-based polymers, ethylene glycol polymers and its copolymers, oxyethylene polymers, polyvinyl alcohol, chitosan and hyaluronan and its copolymers. In an aspect, the carrier agents include hydroxyethyl cellulose, hydroxypropyl cellulose, hydroxypropylmethyl cellulose, hydroxymethyl cellulose, polyvinyl alcohol, polyethylene glycol, polyethylene oxide, ethylene oxide-propylene oxide co-polymers, chitosan, hyaluronan and its copolymers, or combinations thereof. In another aspect, the carrier agents include hyaluronan or hyaluronic acid and copolymers including salts of hyaluronic acid, esters of hyaluronic acid, cross-linked gels of hyaluronic acid, enzymatic derivatives of hyaluronic acid, chemically modified derivatives of hyaluronic acid or combinations thereof. As used herein, hyaluronic acid broadly refers to naturally occurring, microbial and synthetic derivatives of acidic polysaccharides of various molecular weights constituted by residues of D-glucuronic acid polysaccharides and N-acetyl-D-glucosamine.

In certain embodiments, the licorice extract and the carrier agent are in the form of an admixture, in the form of a complex, covalently coupled, or a combination thereof. In certain embodiments, the carrier agent comprises a bioadhesive. Suitable bioadhesive carrier agents include, but are not limited to a cellulose based polymer and/or a dextrin. Suitable cellulose based polymers include, but are not limited to hydroxyethyl cellulose, hydroxypropyl cellulose, hydroxymethyl cellulose, or a mixture thereof. In an embodiment, the bioadhesive carrier agent includes polylactide, polyglycolide, polylactide-co-glycolide, polyethylene glycol, hyaluronan, hyaluronic acid, chitosan, or a mixture thereof. The bioadhesive carrier agent can include a copolymer comprising polyethylene glycol, hyaluronan, hyaluronic acid, chitosan, or a mixture thereof.

In certain embodiments, the carrier agent penetrates periodontal tissues. Suitable penetrating carrier agents include, but are not limited to hyaluronic acid, a hyaluronic acid derivative, chitosan, a chitosan derivative, or a mixture thereof. In an embodiment, the penetrating carrier agent includes a salt of hyaluronic acid, an ester of hyaluronic acid, an enzymatic derivative of hyaluronic acid, a cross-linked gel of hyaluronic acid, a chemically modified derivative of hyaluronic acid, or a mixture thereof.

In certain embodiments, the licorice extract described herein is incorporated into a “home healthcare” formulation. Such formulations include, but are not limited to toothpaste, mouthwash, tooth whitening strips or solutions, dental floss, toothpicks, toothbrush bristles, oral sprays, oral lozenges, and the like.

The formulation of such health products is well known to those of skill, and the licorice extract of the present invention is simply added to such formulations in an effective dose (e.g., a prophylactic dose to inhibit dental caries formation, etc.).

For example, toothpaste formulations are well known to those of skill in the art. Typically such formulations are mixtures of abrasives and surfactants; anticaries agents, such as fluoride; tartar control ingredients, such as tetrasodium pyrophosphate and methyl vinyl ether/maleic anhydride copolymer; pH buffers; humectants, to prevent dry-out and increase the pleasant mouth feel; and binders, to provide consistency and shape (see, e.g., Table 1). Binders keep the solid phase properly suspended in the liquid phase to prevent separation of the liquid phase out of the toothpaste. They also provide body to the dentifrice, especially after extrusion from the tube onto the toothbrush.

TABLE 1
Typical components of toothpaste.

| Ingredients | Wt % |
|---|---|
| Humectants | 40-70 |
| Water | 0-50 |
| Buffers/salts/tartar control | 0.5-10 |
| Organic thickeners (gums) | 0.4-2 |
| Inorganic thickeners | 0-12 |
| Abrasives | 10-50 |
| Actives (e.g., triclosan) | 0.2-1.5 |
| Surfactants | 0.5-2 |
| Flavor and sweetener | 0.8-1.5 |

Fluoride sources provide 1000-15000 ppm fluorine.

Table 2 lists typical ingredients used in formulations; the final combination will depend on factors such as ingredient compatibility and cost, local customs, and desired benefits and quality to be delivered in the product. It will be recognized that the licorice extract of the present invention can simply be added to such formulations or used in place of one or more of the other ingredients.

TABLE 2
List of typical ingredients

| | | | | | Tartar |
|---|---|---|---|---|---|
| | Inorganic | | | | Control |
| Gums | Thickeners | Abrasives | Surfactants | Humectants | Ingredient |
| Sodium | Silica | Hydrated | Sodium | Glycerine | Tetrasodium |
| carboxymethyl | thickeners | silica | lauryl sulfate | | pyrophosphate |
| cellulose | | | | | |
| Cellulose ethers | Sodium | Dicalcium | Sodium N- | Sorbitol | Gantrez S-70 |
| | aluminum | phosphate | lauryl | | |
| | silicates | digydrate | sarcosinate | | |
| Xanthan Gum | Clays | Calcium | Pluronics | Propylene | Sodium tri- |
| | | carbonate | | glycol | polyphosphate |
| Carrageenans | | Sodium | | Xylitol | |
| | | bicarbonate | | | |
| Sodium alginate | | Calcium | Sodium | Polyethylene | |
| | | pyrophosphate | lauryl | glycol | |
| | | | sulfoacetate | | |
| Carbopols | | Alumina | | | |

One illustrative formulation described in U.S. Pat. No. 6,113,887 comprises (1) a water-soluble bactericide selected from the group consisting of pyridinium compounds, quaternary ammonium compounds and biguanide compounds in an amount of 0.001% to 5.0% by weight, based on the total weight of the composition; (2) a cationically-modified hydroxyethylcellulose having an average molecular weight of 1,000,000 or higher in the hydroxyethylcellulose portion thereof and having a cationization degree of 0.05 to 0.5 mol/glucose in an amount of 0.5% to 5.0% by weight, based on the total weight of the composition; (3) a surfactant selected from the group consisting of polyoxyethylene polyoxypropylene block copolymers and alkylolamide compounds in an amount of 0.5% to 13% by weight, based on the total weight of the composition; and (4) a polishing agent of the non-silica type in an amount of 5% to 50% by weight, based on the total weight of the composition. In certain embodiments, the licorice extract of this invention can be used in place of the bactericide or in combination with the bactericide.

Similarly, mouthwash formulations are also well known to those of skill in the art. Thus, for example, mouthwashes containing sodium fluoride are disclosed in U.S. Pat. Nos. 2,913,373, 3,975,514, and 4,548,809, and in US Patent Publications US 2003/0124068 A1, US 2007/0154410 A1, and the like. Mouthwashes containing various alkali metal compounds are also known: sodium benzoate (WO 9409752); alkali metal hypohalite (US 20020114851A1); chlorine dioxide (CN 1222345); alkali metal phosphate (US 2001/0002252 A1, US 2003/0007937 A1); hydrogen sulfate/carbonate (JP 8113519); cetylpyridium chloride (CPC) (see, e.g., U.S. Pat. No. 6,117,417, U.S. Pat. No. 5,948,390, and JP 2004051511). Mouthwashes containing higher alcohol (see, e.g., US 2002/0064505 A1, US 2003/0175216 A1); hydrogen peroxide (see, e.g., CN 1385145); CO₂ gas bubbles (see, e.g., JP 1275521 and JP 2157215) are also known. In certain embodiments, these and other mouthwash formulations can further comprise the licorice extract of this invention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition for reducing dental caries in a human subject, said composition comprising a lollipop or hard candy, wherein said lollipop or hard candy is substantially sugar free, and said lollipop or hard candy comprises an extract from the root of Glycyrrhiza spp., wherein said extract comprises a glycyrrhizol.

2. The composition of claim 1, wherein said lollipop or hard candy is a lollipop.

3. The composition of claim 1, wherein said lollipop or hard candy comprises:
a starch or dextrin;
a flavoring;
a sweetener; and
said extract.

4. The composition of claim 3, wherein said sweetener is non-cariogenic.

5. The composition of claim 3, wherein said sweetener is a sugar alcohol.

6. The composition of claim 3, wherein said sweetener is an artificial sweetener.

7. The composition of claim 3, wherein said sweetener is selected from the group consisting of stevia, aspartame, saccharin, acesulfame k, sucralose, and neotame.

8. The composition of claim 3, wherein said lollipop or hard candy comprises:
hydrogenated starch hydrolysate;
citric acid;
flavoring; and
acesulfame potassium.

9. The composition of claim 8, wherein said lollipop or hard candy further comprises a coloring agent.

10. The composition of claim 1, wherein said extract comprises substantially purified glycyrrhizol A, and/or substantially purified glycyrrhizol B, and/or substantially purified 6,8-diisoprenyl-5,7,4′-trihydroxyisoflavone.

11. The composition of claim 10, wherein said extract comprises substantially purified glycyrrhizol A.

12. The composition of claim 1, wherein said extract consists essentially of substantially purified glycyrrhizol A and/or substantially purified glycyrrhizol B, and/or substantially purified 6,8-diisoprenyl-5,7,4′-trihydroxyisoflavone.

13. The composition of claim 12, wherein said extract consists essentially of substantially purified glycyrrhizol A.

14. The composition of claim 1, wherein said lollipop or hard candy contains from about 2 to about 200 mg of said extract.

15. The composition of claim 1, wherein said lollipop or hard candy contains from about 2 mg to about 100 mg of said extract.

16. The composition of claim 1, wherein said lollipop or hard candy contains from about 2 to about 30 mg of said extract.

17. The composition of claim 15, wherein said hard candy or lollipop is formulated to dissolve in about 2 minutes to about 15 minutes in a human mouth.

18. The composition of claim 15, wherein said hard candy or lollipop is formulated to take at least about 3 minutes.

19. The composition of claim 15, wherein said extract is produced according to claim 29.

20. A method of reducing the incidence of dental caries in a human, said method comprising administering to said human a composition that delivers to the oral cavity of said human a glycyrrhizol, wherein said delivery is for a period of at least about 4 minutes.

21. The method of claim 20, wherein said period is at least 5 minutes.

22. The method of claim 20, wherein said period ranges from about 5 minutes to about 15 minutes.

23. The method of claim 20, wherein said glycyrrhizol is provided as an extract from the root of Glycyrrhiza spp.

24. The method of claim 20, wherein said extract comprises substantially purified glycyrrhizol A.

25. The method of claim 20, wherein said extract consists essentially of substantially purified glycyrrhizol A.

26. The method of claim 23, wherein about 2, 5, or 10 mg to about 100, 50, 25, 20, or 15 mg of said extract is delivered to the oral cavity.

27. The method of claim 20, wherein said composition comprises a lollipop containing glycyrrhizol.

28. The method of claim 20, wherein said composition comprises a lollipop or hard candy according to claim 1.

29. A method of providing an extract of licorice that inhibits the formation of dental caries by Streptococcus mutans, said method comprising:
providing root of Glycyrrhiza spp.
performing an alcohol extraction of said root; and
drying the extract to produce an extract that has activity against S. mutans.

30. The method of claim 29, wherein said root is cut to pieces having an average thickness of about 2 mm or less, or about 1 mm or less.

31. The method of claim 29, wherein said root is grown and harvested in inner Mongolia.

32. The method of claim 29, wherein said alcohol extraction comprises a first extraction combining the root with 95% food grade ethanol in a 1:8 ratio.

33. The method of claim 32, wherein the root/ethanol mixture is incubated for about 72 hours.

34. The method of claim 32, further comprising removing the ethanol, and performing a second extraction by adding fresh 95% food grade ethanol in about a 1:8 ratio and incubating again.

35. The method of claim 32, wherein said ethanol is dried to produce a substantially dry powder extract.

36. The method of claim 29, wherein said method does not comprise solvent/solvent partitioning using an organic solvent.

37. A composition for reducing dental caries in a human subject, said composition comprising:
a licorice extract produced according to claim 29.

38. A composition for reducing dental caries in a human subject, said composition comprising a licorice extract according to claim 37 wherein said licorice extract is contained in a product selected from the group consisting of toothpaste, mouthwash, a tooth whitening strip or solution, dental floss, a toothpick, a toothbrush bristle, an oral spray, an oral lozenge, and an aerosolizer for oral application.
